(19) United States
(12) Patent Application Publication
(10) Pub. No.: US 2007/0012340 A1
(43) Pub. Date: Jan. 18, 2007

(54) System and method for cleaning or sanitizing items intended for re-use

(75) Inventors: W. Scott Jones, Carrollton, GA (US); Tobin R. King, Carrollton, GA (US); Carter L. Jones, Rome, GA (US)

Correspondence Address: MICHAEL E. MCKEE;Attorney at Law, 804 Swaps Lane, Knoxville, TN, 37923, US

(73) Assignee: Environmental Cart Cleaning, LLC

(21) Appl. No.: 11/183,539

(22) Filed: Jul. 18, 2005

(51) Int. Cl. B08B 3/00

(52) U.S. Cl. 134/45; 134/57 R; 134/131; 134/123

(57) ABSTRACT

A system and method for sanitizing or cleaning an item, such as a grocery store shopping cart or a child stroller, for re-use utilizes a passageway having an entrance and an exit and a sanitizing or cleaning station between the entrance and exit. The item is conveyed through the passageway into the station, and pulsating beams of ultraviolet (UV) light are directed toward the item to sanitize the surfaces of the item which are struck by the pulsating beams of light. The system can be supplemented with an arrangement for directing water toward the item for rinsing purposes and for subsequently directing air toward the item for drying purposes. Moreover, the rinse water can be collected and sanitized after it has been directed toward the item during a rinsing operation for subsequent re-use.

### BACKGROUND OF THE INVENTION

This invention relates generally to the cleaning or sanitizing of surfaces and relates, more particularly, to means and methods for sanitizing items which are commonly exposed to bacteria yet are intended to be used repeatedly.

The class of items with which this invention is concerned includes shopping carts and child strollers, such as those which are employed by individuals for either collecting food items within a grocery store for purchase or for transporting children through a store or theme park. Such items are also expected to be re-used by others after use.

Items of this class includes surfaces which, during use, might be exposed to undesirable material, such as the drippings from raw chicken and other meats or dirty diapers worn by children who sit within such an item, or might be contacted by the unwashed hands of an individual who uses such an item while he is sick. If left uncleaned after use, the exposed surfaces of such an item could spawn the growth of unwanted micro-cultures, such as salmonella, e coli, botulism and enteroccocus facaelis, to which a subsequent user of the item could be exposed.

With regard to grocery store shopping carts, efforts have been made to clean the carts with a steam wand in the store parking lot, but any bacteria which is washed from the cart surfaces, if left untreated, could migrate into and pollute a public water source (e.g. a river, lake, stream or ocean). Some grocery stores have made sanitized wipes available which permit a customer to manually wipe a shopping cart clean before it is used, but the amount of time and effort normally required to adequately clean a shopping cart in this manner often discourages the customer from cleaning the shopping cart at all.

It would therefore be desirable to provide an improved means and associated method for sanitizing or cleaning an item of the aforedescribed class which requires relatively little time and effort to operate or carry out and which reduces the likelihood that harmful bacteria which could be spawned upon the container surfaces would migrate to the public water supply following a sanitizing or cleaning operation.

Accordingly, it is an object of the present invention to provide a new and improved system and method for sanitizing or cleaning a food-collection container, such as a grocery store shopping cart or a child stroller.

Another object of the present invention is to provide such a system which does not require that the surfaces of the item be manually rubbed or wiped during the sanitizing process.

Still another object of the present invention is to provide such a system whose operation is automatic and requires relatively little time or effort to sanitize and clean an item.

A further object of the present invention is to provide such a system which is particularly well-suited for sanitizing or cleaning grocery store shopping carts and child strollers.

A still further object of the present invention is to provide such a system which is uncomplicated in structure, yet effective in operation.

### SUMMARY OF THE INVENTION

This invention resides in a system and method for sanitizing an item, such as a grocery store shopping cart or a stroller, for re-use.

The system of the invention includes means defining a passageway having an entrance through which an item to be sanitized enters the passagway-defining means, an exit through which the item exits the passageway-defining means, and a passageway station disposed between the entrance and the exit of the passageway. In addition, there are provided means for conveying the item between the entrance and the exit of the passageway so that the item is conveyed through the passageway station. Furthermore, the system includes means associated with the passageway station for directing beams of light toward the item as the item is positioned within the passageway station so that the surfaces which are struck by the beams of light are sanitized thereby.

In a particular embodiment of the system, the system includes means associated with the passageway station for directing a liquid medium toward the item as the item is positioned within the passageway station so that the surfaces which are struck by the liquid medium are cleaned thereby, and means are connected to the passageway-defining means for re-circulating the liquid medium through the passageway station so that after the liquid medium has been directed toward the item, the liquid medium is collected for purposes of being subsequently directed toward an item positioned within the passageway station.

The method of the invention includes the steps performed by the system of the invention. In particular, such steps include the providing of a passageway having an entrance through which an item to be sanitized enters the passageway-defining means, an exit through which the item exits the passageway-defining means, and a passageway station disposed between the entrance and the exit of the passageway. The item is then conveyed between the entrance and the exit of the passageway so that the item is conveyed through the passageway station, and beams of ultraviolet (UV) light are directed toward the item as the item is positioned within the passageway station so that the surfaces which are struck by the beams of UV light are sanitized thereby.

### BRIEF DESCRIPTION OF THE DRAWINGS

FIG. 1 is a perspective view, shown partially cut-away, of an embodiment of a sanitizing and cleaning system within which features of the present invention are embodied.

FIG. 2 is a side elevational view, shown partially cut-away, of the FIG. 1 system and a plurality of shopping carts being advanced through the system.

FIG. 3 is a fragmentary end view of the FIG. 1 system as seen generally from the left in FIG. 1 and illustrating the entrance door of the FIG. 1 system in an opened condition.

FIG. 4 is a view similar to that of FIG. 3 and illustrating the entrance door of the FIG. 1 system in a closed condition.

FIG. 5 is a view which schematically illustrates in block diagram form the operation of several components of the FIG. 1 system.

### DETAILED DESCRIPTION OF AN ILLUSTRATIVE EMBODIMENT

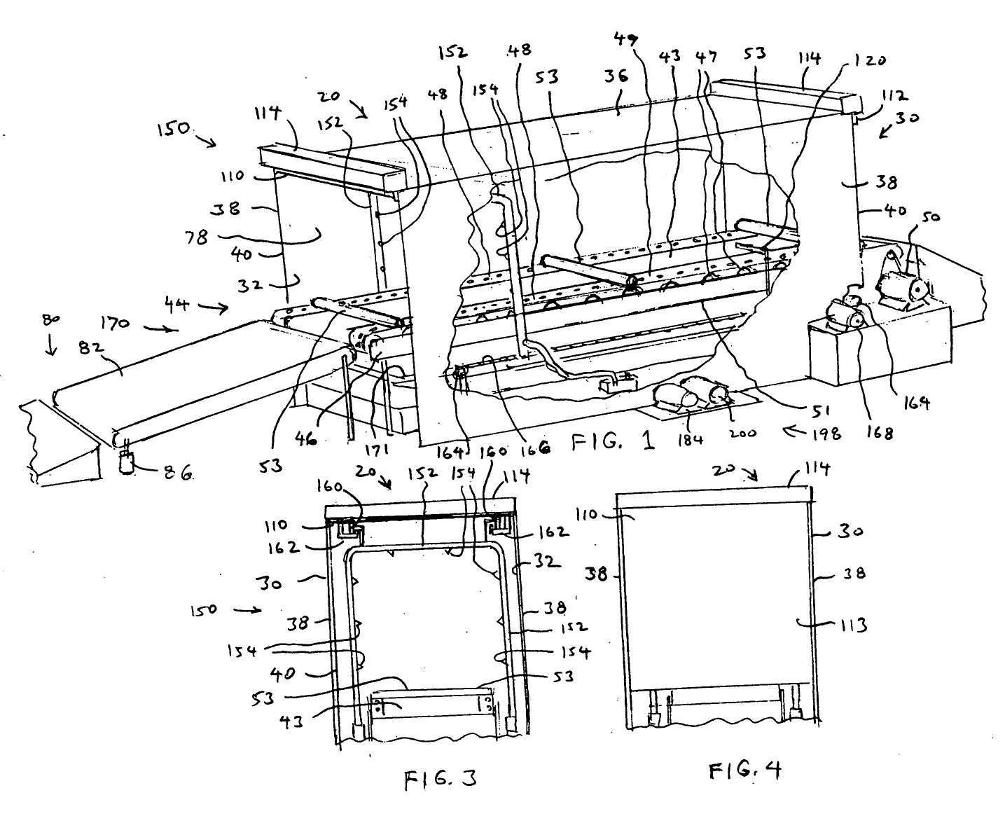

Turning now to the drawings in greater detail and considering first FIGS. 1 and 2, there is illustrated an embodiment, generally indicated 20, of a sanitizing system within which features of the present invention are embodied and which can be used to sanitize and clean the surfaces of items 22 as the items 22 are advanced through the system 20. The item 22 intended to be sanitized with the depicted system 20 is a shopping cart 23 which is commonly used by a grocery store customer to collect food items for purchase. As is common with grocery store shopping carts and as best shown in FIG. 2, each shopping cart 23 includes front and rear wheels 28 and 29, respectively, and a container section 24 having a bottom and sidewalls comprised of grillwork which can be constructed of metal, plastic or other suitable material.

During use of the shopping cart 23, undesirable materials might be transferred to the surfaces of the container section 24 by selected items of food or by the customer or his children. For example, during use of the shopping cart 23, its surfaces might be exposed to the drippings from raw chicken and other meats or even to the dirty diapers worn by children who are permitted sit within the shopping cart 23. Further still, the shopping cart surfaces might be contacted by the unwashed hands of a customer who uses the shopping cart 23 while he is sick. Of course, if such surfaces of the shopping cart 23 are left uncleaned after use, any unwanted material transferred thereto could spawn the growth of unwanted micro-cultures, such as salmonella, e coli, botulism and enteroccocus facaelis, to which a subsequent user of the shopping cart 23 could be exposed.

As will be apparent herein, the system 20 can be used to quickly and effectively sanitize and clean the surfaces of the depicted shopping cart 23 after each use so that the depicted shopping cart 23 is relatively free of active undesirable microorganisms for use by a subsequent customer. In this connection, the operation of the system 20 employs a sanitizing stage, a rinsing (e.g. a cleaning) stage, and a drying stage, and the various stages of system operation are automatically controlled by a computer controller 119 (FIG. 5) so that once the operation of the system 20 is initiated, no operator intervention is necessary.

With reference again to FIGS. 1 and 2, the system 20 includes means, generally indicated 30, defining an elongated passageway 32 through which a plurality of shopping carts 23 are automatically moved in preparation of and upon completion of a sanitizing operation performed with the system 20. The passageway-defining means 30 of the depicted system 20 includes a top 36 and two opposite sidewalls 38 which extend between the top 36 and the floor 33 or similar underlying support surface. One end, indicated 40, of the passageway 32 provides an entrance for the passageway-defining means 30, and the opposite end, designated 42, of the passageway 32 provides an exit for the passageway-defining means 30. As will be apparent herein, shopping carts 23 which are desired to be sanitized within the system 20 are directed in an end-to-end fashion into the passageway 32 at the entrance 40 thereof, and upon completion of the sanitizing and cleaning operation, the shopping carts 23 exit the passageway 22 at the exit 42 thereof.

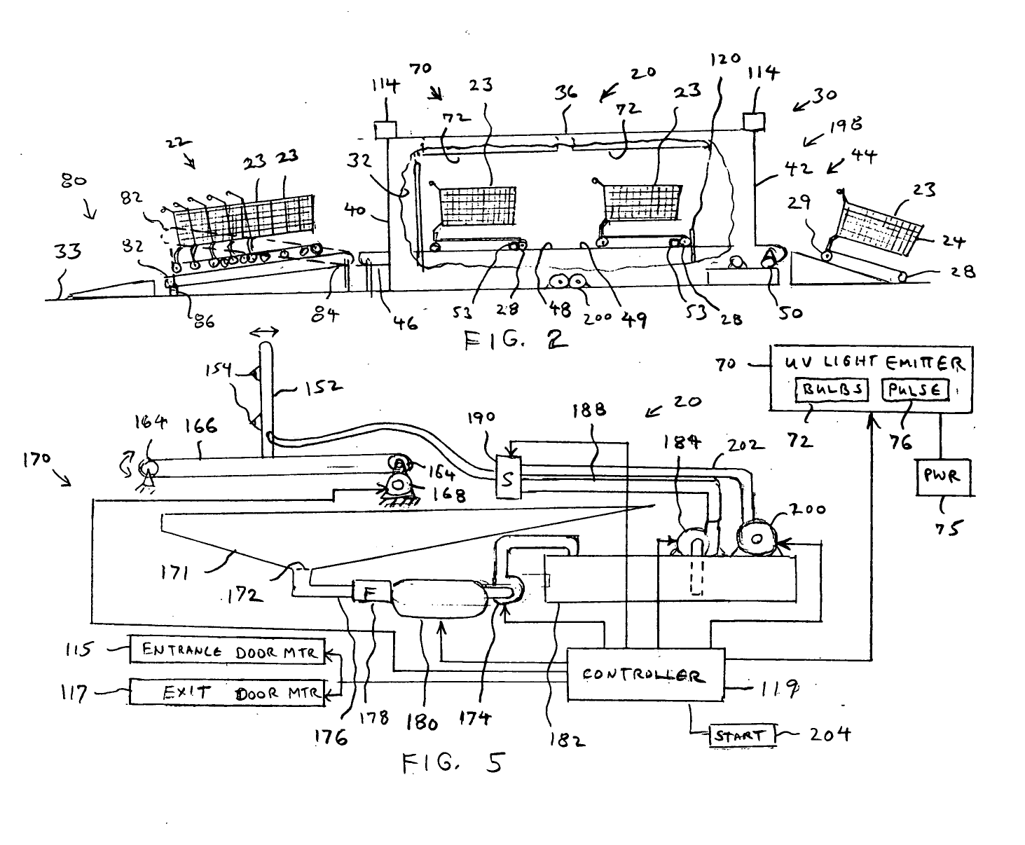

With reference to FIGS. 1-4, the passageway-defining means 30 includes an entrance door 110 adjacent the passageway entrance 40 and includes an exit door 112 (FIG. 2) adjacent the passageway exit 42. Each door 110 and 112 is in the form of an electrically-powered roll-up door which, as is exemplified by the flexible closure member 113 of the entrance door 110 of FIGS. 3 and 4, is movable from an opened condition at which the carts 23 are permitted to pass through the passageway entrance 40 or exit 42 and a closed condition at which each of the entrance 40 and exit 42 of the passageway 32 is closed off. When in the opened condition, the closure member for each door 110 or 112, as well as the associated reversible motor 115 or 117 (FIG. 5) for moving the doors 110 and 112 between opened and closed conditions, is housed in an opened-bottom enclosure 114 mounted on top of the passageway 32 adjacent a corresponding end 40 or 42 thereof. Doors which are suitable for use as the doors 110 and 112 are available from Albany Door Systems, Inc. Of Lawrenceville, Georgia under the trade designation Rapid Roll.

For purposes of moving the shopping carts 23 through the passageway 32, the system 20 includes conveyor means, generally indicted 44 in FIGS. 1 and 2, for conveying the shopping carts 23 through the passageway 32 from the entrance end 40 to the exit end 42 thereof. In this connection, the conveyor means 44 includes an elongated frame 46 which extends between the ends 40 and 42 of the passageway 32 and a stationary support surface 43 which extends along the entire length of the passageway 32, and a plurality of sprockets 47 are rotatably mounted along each side of the support surface 43. An endless conveyor chain 48 is looped about the sprockets 47 disposed along each side of the support surface 43 so that an upper section, indicated 49, of the conveyor chain 48 is movable in one linear direction (e.g. toward the right as illustrated in FIGS. 1 and 2) relative to the frame 46 while a lower section, indicated 51 in FIG. 1, of the chain 48 is movable in the opposite linear direction (e.g. toward the left as illustrated in FIG. 1). In addition, a plurality of roller members 53 extend transversely across the chain 48 (so as to span the support surface 43 when moved therealong) and are each rotatably secured at each end to the chain 48.

For advancing the upper section 49 of the conveyor chain 48 from the passageway entrance 40 to the passageway exit 42, an electrically-powered motor 50 is mounted at the frame end corresponding with the passageway exit 42 and is connected in driving relationship with the sprockets 47 disposed adjacent the exit 42 of the passageway 32 whose teeth are, in turn, positioned in meshed relationship with the conveyor chain 48. Therefore, when the motor 50 is energized, the upper section 49 of the chain 48 is moved from the entrance end 40 of the passageway 32 toward the exit end 42 thereof while the rollers 53 are advanced along the support surface 43.

During operation of the conveyor means 44 and when conveyed onto the support surface 43 adjacent the passageway entrance 40, a roller member 53 abuts, and thereby catches, the back of the front wheels 28 of a shopping cart 23 appropriately positioned upon the conveyor belt 48 adjacent the passageway entrance 40 so that the front wheels 28 of the cart 23 are pushed along the support surface 43 toward the passageway exit 42 by the roller member 53. Although the back of the front wheels 28 are engaged by a roller member 53 as the cart 23 is pushed along the support surface 43 by the roller member 53, the capacity of the roller member 53 to rotate about its longitudinal axis permits the front wheels 28 of the cart 23 to freely roll (forwardly) along the support surface 43 as the roller members 53 are rotated by the cart wheels 28 in a rotational direction opposite the rotational direction in which the cart wheels 28 are rotated.

To facilitate the advancement of the carts 23 onto the stationary support surface 43 adjacent the passageway entrance 40, there is associated with the system 20 a ramp, generally indicated 80, having a hinged section 82. The hinged section 82 is pivotally supported at one end, indicated 84, for pivotal movement between the position illustrated in solid lines FIG. 2 at which the carts 23 can be loaded upon the section 82 and the position illustrated in phantom in FIG. 2, at which the carts 23 are permitted to gravitationally move (i.e. roll) toward the passageway entrance 40. For moving the ramp section 82 between the FIG. 2 solid and phantom-line positions, there is provided an electrically-powered jack 86 having a ram which is connected to the ramp section 82 opposite the end 84 thereof and whose operation can be controlled by an operator.

It follows that as each shopping cart 23 is directed onto the support surface 43 adjacent the passageway entrance end 40 and the conveyor motor 50 is energized, each roller member 53 which is advanced onto the support surface 43 by the chain 48 abuts and thereby catches the front wheels 28 of a corresponding cart 23 and moves (i.e. pushes) the shopping cart 23 along the length of the passageway 32. It also follows that the conveyor means 44 is of sufficient size and strength to suitably transport shopping carts 23 which are placed upon the support surface 43 along the length of the conveyor frame 46, and that the spacing between adjacent shopping carts 23 conveyed through the passageway 32 can be controlled by spacing the roller members 53 associated with the chain 48 an appropriate, or preselected, distance apart.

As is addressed in greater detail herein, the conveyor means 44 is adapted to halt the advance of a shopping cart 23 when the cart 23 has advanced a predetermined distance into the passageway 32 so that a sanitizing and cleaning operation can be performed upon the shopping cart 23 while the cart 23 is disposed in a stationary condition. In this connection, there is associated with the system 20 a movable switch mechanism 120 (FIG. 2) which, when engaged by the front of a cart 23, sends an appropriate signal to the computer controller 119 (FIG. 5) which, in turn, de-energizes the conveyor motor 50 and initiates the system operation to be performed upon the cart 23.

As mentioned earlier, the carts 23 which are positioned within the passageway 32 undergo a sanitizing stage, a rinsing (or cleaning) stage, and a drying stage. In this connection and with reference again to FIG. 2, there is provided between the passageway entrance 40 and exit 42 a workstation, or station 62, within which two carts 23 are positioned and within which the various system operations are performed upon the carts 23. Within the depicted system 20, the station 62 possesses such a size and length to accommodate two carts 23 positioned therein in an end-to-end arrangement. Accordingly and within the system 20, the various stages of the system operation can be performed upon two carts 23 at the same time. It will be understood, however, that a system embodiment whose passageway station accommodates only one cart 23 positioned therein or whose passageway station accommodates more than two carts 23 positioned therein can be had. Accordingly, the principles of the present invention can be variously applied.

For purposes of sanitizing the shopping carts 23 (during a sanitizing stage of system operation) as the carts 23 are disposed within the passageway station 62 and with reference still to FIG. 2, the system 22 includes means, generally indicated 70, for directing, or emitting, light beams in a pulsating manner toward the shopping carts 23 for sanitizing the surfaces of the shopping carts 23 which are exposed to the pulsating beams of light. To this end, the light-emitting means 70 includes a pair of elongated light bulbs 72 which are mounted beneath the top 36 of the passageway 32 and which are disposed in an end-to-end arrangement along the length of the station 62. In addition, the bulbs 72 are directed downwardly toward the carts 23 positioned within the station 62. Each bulb 72 is adapted to emit beams of light having wavelengths which appreciably fall within the ultraviolet (UV) range and which is known to kill bacteria when struck by such UV beams. Associated with the light bulbs 72 is a electrical power supply 75 (FIG. 5) for energizing the bulbs 72 and electronic means 76 for intermittently switching the light bulbs 72 ON and OFF in a pulsating fashion so that the bulbs 72 are never energized continuously for a lengthy period of time. In practice, the surfaces of the carts 23 can be satisfactorily sanitized within only a few seconds of exposure to the beams emitted from the bulbs 72.

To enhance the sanitizing effects of the pulsating beams of light emitted from the light bulbs 72, the sidewalls 38 of the passageway-defining means 30 are preferably lined with a reflective material, such as highly-polished aluminum sheeting, indicated 78 in FIG. 1, for reflecting the pulsating beams of light which strike the reflective sheeting 78 inwardly of the sanitizing station 62 (e.g. back toward the shopping carts 23). The reflective sheeting 78 thereby increases the likelihood that surfaces of the shopping cart 23 which are not directly in the line of sight of the pulsating beams emitted from the bulbs 72 will be still be exposed to beams which are reflected from the sheeting 78. Along the same lines and if desired, the interior, or back side surface, of the closure members of the entrance and exit doors 110 and 112 can be lined with reflective material, as well.

Light bulbs which are suited for use as the light bulbs 72 are available from Xenon Corporation of Woburn, Mass.

Upon completion of the sanitizing stage of the system operation (during which the surfaces of the carts 23 are exposed to the light beams emitted by the bulbs 72), the carts are then rinsed and cleaned in a rinsing operation. In this connection, the system 20 includes means, generally indicated 150, for directing a liquid cleaning or rinsing medium (e.g. water) toward the shopping carts 23 while the carts 23 are positioned within the station 62. More specifically, the liquid medium-directing means 150 includes a U-shaped spray manifold 152 (FIGS. 1 and 3) which is positioned within the sanitizing station 62 so as to extend upwardly along the sides of and across the top of the carts 23 positioned within the station 62. The spray manifold 152 includes a plurality of spray nozzles 154 which are directed toward the interior of the station 62 so that a liquid medium (e.g. water) which is delivered to the nozzles 154 in a pressurized condition (e.g. about 250 psi) is directed out of the nozzles 154 and sprayed over the surfaces of the carts 23 positioned within the station 62. As the liquid medium which is directed out of the nozzles 154 strikes the carts 23, contaminants which may happen to cling to the surfaces of the carts 23 from a previous use are likely to be dislodged and rinsed from the surfaces of the cart 23.

In addition, the manifold 152 is movably supported within the station 62 for movement between the passageway ends 40 and 42 thereof during a sanitizing operation performed with the system 20. In this connection and with reference to FIG. 3, a pair of wheels 160 are rotatably connected to the manifold 152 adjacent the upper end thereof, and these wheels 160 are supported within a pair of guide tracks 162 which extend along the entire length of the station 62 between the passageway ends 40 and 42. Furthermore, the lower ends of the manifold 152 are connected to a drive chain 166 (FIG. 1) which extends along the length of the passageway 32 and which is rotatably supported at each end by a pair of sprockets 164 which are rotatably supported adjacent a corresponding end 40 or 42 of the passageway 32. A reversible drive motor 168 (FIG. 1) is drivingly connected to the drive chain 166 so that when the motor 168 is actuated so that its drive shaft is rotated in one rotational direction, the manifold 152 is moved (by way of the drive chain 166) in one direction (e.g. from the passageway entrance 40 toward the passageway exit 42) along the length of the passageway 32, and so that when the motor 168 is actuated so that its drive shaft is rotated in the opposite rotational direction, the manifold 152 is moved (by way of the drive chain 166) in the opposite direction (e.g. from the passageway entrance 40 toward the passageway exit 42) along the length of the passageway 32. It will be understood that as the manifold 152 is moved between the passageway entrance 40 and exit 42, the wheels 160 are guided and rolled along the guide tracks 162.

Although the manifold 152 has been described within the depicted system 20 as being mounted for rolling movement between the passageway entrance 40 and exit 42 by way of a wheel and guide track arrangement, the manifold 152 can be slidably moved between the entrance 40 and the exit 42 by way of a pillow block and guide rail arrangement. In this latter case, for example, the manifold 152 is attached to pillow blocks which are, in turn, slidably mounted upon guide rails which extend between the passageway entrance 40 and exit 42.

For purposes of conserving the liquid medium (e.g. water) which is directed over the carts 23 during the rinsing stage of system operation, the system 20 includes a re-circulation system, generally indicated 170 in FIGS. 1 and 5, which re-circulates liquid medium back through the system 20 where it can be re-directed toward the carts 23 positioned within the passageway station 62. Within the system 20, the re-circulation system 170 includes a collection tray 171 positioned within the station 62 and beneath the support surface 43 for gravitationally collecting the liquid medium after it has been directed toward the carts 23. Once collected within the tray 171, the liquid medium is permitted to gravitationally flow toward a drain 172 (FIG. 5) disposed at the bottom of the tray 171. From the drain 172, the liquid medium is pumped (by means of a re-circulation pump 174) along a conduit arrangement 176 which includes an internal (e.g. micron) filter 178 for collecting contaminants which may have been dislodged and rinsed from the carts 23 and a purification chamber 180 through which the liquid medium is routed before being collected within a reservoir 182 disposed beneath the collection tray 171. Liquid medium which is collected within the reservoir 182 can be withdrawn therefrom with a pump 184 returned to the manifold 152, by way of a hose 188 and a solenoid valve 190 described herein, where the liquid medium can be re-directed toward the carts 23 situated within the station 62.

The purification chamber 180 includes a light bulb which is adapted to generate and direct light beams toward the liquid medium routed through the conduit arrangement 176 so that the light beams kill bacteria within and thereby sanitize the liquid medium. In this connection, the wavelengths of the light beams which are generated within the purification chamber 180 include frequencies which appreciably fall within the ultraviolet (UV) range which are known to kill bacteria struck by the generated light beams. Purification chambers which are adapted to kill bacteria in water flowing therethrough and which are suitable for use as the chamber 180 are manufactured by and are commercially available from R-Can Environmental Inc. of Guelph, Ontario, Canada.

Upon completion of the rinsing stage (during which the cart surfaces are rinsed with the liquid medium directed thereon by the liquid medium-directing means 150), the carts 23 are exposed to air blown toward the carts 23 in a drying stage. In this connection, the system 20 includes air-directing means, generally indicated 198, including a compressor 200 which is connected to the solenoid valve 190, introduced earlier, by way of an air hose 202 for directing air therethrough to the manifold 152. The solenoid valve 190 is a two-position valve so that when (the internal switch mechanism of) the valve 190 is in a first position, the liquid medium which is pumped from the pump 184 is permitted to pass through the valve 190 to the manifold 152 where the liquid medium is directed out of the nozzles 154 and toward the carts 23 positioned within the station 62 and so that when (the internal switch mechanism of) the valve 190 is in a second position, the air which is pumped from the air compressor 200 is permitted to pass through the valve 190 to the manifold 152 where the air is directed out of the toward the carts 23 positioned within the station 62.

During the operation of the system 20 and, in particular, during the drying cycle thereof, air is directed out of the manifold nozzles 154 as the manifold 152 is moved (by way of the drive chain motor 160) from the passageway exit 42 toward the passageway entrance 40. Furthermore and since the operation of the system 20 does not require that the liquid medium be directed out of the manifold 152 at the same time that the air is directed out of the manifold 152 and vise-versa, the solenoid valve 190 permits the manifold 152 to be alternatively used during a rinse cycle and during a drying cycle of the system operation and is advantageous in this respect.

With reference to FIG. 5, the computer controller 119, introduced earlier, is connected to each of the principal components of the system 20 for automatically controlling the operation and sequencing of the various system components. At the outset of a cycle of operation performed with the system 20, the entrance and exit doors 110, 112 can be disposed in an open condition, although it may be desirable to begin a cycle of system operation with the doors 110, 112 disposed in a closed condition for purposes of safety or security of the system 20. Moreover and at the outset of a cycle of operation of the system 20, the manifold 152 is disposed adjacent the entrance end 40 of the passageway 32. For exemplary purposes, a cycle of system operation will now be described with the entrance and exit doors 110, 112 disposed in a closed condition at the outset of a cycle of system operation.

To initiate the system operation, at least one cart 23 or a stack of carts 23 is manually positioned upon the hinged section 82 of the ramp 80, and an operator manually presses a START button 204 (FIG. 5) associated with the computer controller 119 so that command signals are sent to the motors 115, 117 associated with the entrance and exit doors 110, 112 so that the closure members of the doors 110, 112 are raised to a opened condition. The movement of the conveyor chain 48 (by way of the drive motor 50) is then initiated so that two of the roller members 53 catch, in sequence, two of the carts 23 and move (i.e. push) these carts 23 through the station 62 and toward the passageway exit 42. When the forwardmost cart 23 is advanced to and contacts the switch 120, the conveyor chain 48 halts any further advance of the carts 23 so that the both of the carts 23 are positioned in a stationary condition within the station 62 for being acted upon during the sanitizing, rinsing and drying stages of the system operation.

As soon as the advance of the carts 23 through the station 62 is halted by the switch 120, the entrance and exit doors 110, 112 are moved, by way of the door motors 115, 117 to a closed condition to substantially enclose the carts 23 within the station 62. At that point, the light beam-directing means 70 are actuated so that a sufficient amount of UV light is directed toward the carts 23 from the light bulbs 70 in order to kill the bacteria, and thereby sanitize, disposed upon the surfaces of the carts 23. In practice, only a few light pulses of the light bulbs 72 are ordinarily necessary to kill the bacteria upon the surface of the carts 23, and the reflective material 78 disposed upon the interior surfaces of the station 62 increases the likelihood that substantially all of the exposed surfaces of the carts 23 are struck by the beams of UV light.

Upon completion of the sanitizing stage with the UV light, the rinse stage begins. In this connection, appropriate command signals are sent to the pump 184 from the computer controller 119 so that a liquid medium (e.g. water) is pumped from the reservoir 182, conducted through the solenoid valve 190, into the manifold 152 and out of the nozzles 154 toward the carts 23 situated within the station 62. The two-position solenoid valve 190 is normally set to route water from the pump 184 toward the manifold 152 (rather than route air from the compressor 200 toward the manifold 152) so that no adjustment of the valve 190 is necessary prior to the initiation of the rinsing stage. In addition and in conjunction with the directing of the liquid medium (e.g. water) out of the manifold nozzles 154, the drive motor 166 is actuated so that the manifold 152 is bodily moved along the length of the passageway 32 toward the exit 42 thereof as the drive chain 166, by means of the drive motor 168, advances the lower end of the manifold 152 toward the exit 42 and the wheels 160 associated with the upper end of the manifold 152 are guided along the length of the guide tracks 162.

It follows that as the manifold 152 is bodily moved from the passageway entrance 40 toward the passageway exit 42 and the liquid medium continues to be directed (e.g. sprayed) toward the carts 23, the effectiveness of the directed liquid medium to dislodge and rinse contaminants from the surfaces of the carts 23 is improved. When the manifold 152 is eventually moved to a position adjacent the passageway exit 42 (corresponding to the rightwardmost limit of travel of the manifold 152) and the wheels 160 reach the end of the guide track 162 adjacent the passageway exit 42, the rinse stage is complete and any continued advance of the drive chain 166 toward the passageway exit 42 is halted by the computer controller 119 and the pump 184 is de-energized.

Liquid medium which is directed toward the carts 23 through the manifold nozzles 154 is permitted to gravitate downwardly into the collection tray 171 and then out of the tray 171 through the drain 172. As soon as a rinse stage is underway, the re-circulation pump 174 is energized (by way of the controller 119) so that any liquid medium which exits the collection tray 171 through the drain 172 is conducted through the filter 178 and then through the purification chamber 180 where the liquid medium which is pumped therethrough is sanitized by the beams of UV light generated within the purification chamber 180. If desired, the operation of the purification chamber 180 can be coordinated with the operation of the re-circulation pump 174 so that beams of UV light are generated within the purification chamber 180 for sanitizing purposes only when the pump 174 is operated. Once the liquid medium has passed through the purification chamber 180, it is conducted into the reservoir 182 for re-use or, in other words, returned to the quantity of liquid medium from which liquid medium is withdrawn by the pump 184 for cart-rinsing purposes.

Upon completion of a rinsing stage, the control computer 119 begins the drying stage by energizing the air compressor 200 and actuating the solenoid valve 190 so that air which is pumped through the compressor 200 is routed through the solenoid valve 190 and toward the manifold 152 where it is directed out of the nozzles 154 toward the carts 23 to help dry the carts 23. Furthermore, appropriate command signals are sent from the computer controller 119 to the drive motor 168 so that the drive chain 166 begins to advance from the passageway exit 42 toward the entrance 40 thereof and so that the manifold 152 is thereby bodily moved from the exit 42 toward the entrance 40. It follows that as the manifold 152 is moved toward the entrance 40 by the drive motor 168 during the drying stage, air continues to be directed toward the carts 23 through the manifold nozzles 154 to blow the liquid medium from the surfaces of the carts 23 and thereby dry the carts 23. Since the manifold 152 is filled with liquid medium at the outset of a drying stage, it may be desirable to incorporate within the computer controller 119 a time delay which delays the operation of the drive motor 168 (and consequently the movement of the drive chain 166 toward the passageway entrance 40) until a few seconds (e.g. five seconds) have passed from the moment that air begins to be conducted into the manifold 152 through the solenoid valve 190. This way, the manifold 152 is purged of liquid medium by the compressor-pumped air before the manifold 152 begins to be advanced toward the passageway entrance 40.

When the manifold 152 is eventually returned to a position adjacent the passageway entrance 40 at which the wheels 160 reach the end of the guide track 162 adjacent the entrance 40 (corresponding to the leftwardmost limit of travel, as seen in FIGS. 1 and 2, of the manifold 152), the drying stage is complete and any continued advance of the drive chain 166 toward the passageway entrance 40 is halted by the computer controller 119, the compressor 200 is de-energized, and the solenoid valve 190 is de-actuated in preparation of the next rinsing cycle to be performed with the system 20. In addition and upon completion of the drying stage, the entrance and exit doors 110, 112 are opened, and the conveyor motor 48 is energized to advance the sanitized and cleaned carts 23 out of the passageway exit 42 and advance the next two carts 23 (which are positioned upon the ramp section 82) into the station 62 in preparation of subsequent sanitizing, rinsing and drying stages to be performed within the station 62.

It follows from the foregoing that a system 20 and method have been described for sanitizing and cleaning at least one grocery store shopping cart 23 for re-use. The sanitizing of the shopping cart surfaces is effected with light bulbs 72 which are adapted to generate and emit beams of UV light for the purpose of killing the bacteria struck by the UV light beams. Because the interior surfaces of the passageway 32 are covered with a material 78 which is highly reflective to the UV light, the effectiveness of the UV light beams to strike all of the exposed surfaces of the cart 23 is enhanced.

Upon completion of a sanitizing stage of the system operation, a rinsing stage is initiated wherein a liquid medium, such as water, is directed toward the shopping carts 23 positioned within the station 62 for dislodging and rinsing contaminants which might be clinging to the cart surfaces, and a re-circulation system 170 collects and sanitizes the liquid medium which has been directed onto the carts 23 and pumps the sanitized liquid medium to a reservoir 182 for subsequent use. Upon completion of the rinsing stage, a drying stage is initiated wherein air is directed toward the carts 23 to help dry the carts 23. Upon completion of the drying stage, the carts 23 are automatically conveyed from the system 20, and additional carts 23 to be cleaned are moved into the station 62 for a subsequent cleaning operation.

It will be understood that numerous modifications and substitutions can be had to the aforedescribed embodiment without departing from the spirit of the invention. For example, although the aforedescribed system embodiment 20 has been shown and described as being utilized for sanitizing wheeled grocery store shopping carts 23, the system embodiment 20 is also well-suited for sanitizing and cleaning child strollers for re-use.

Further still, although the aforedescribed embodiment has been shown and described as including air-directing means 198 which utilize the manifold 152 and nozzles 154 used by the liquid medium-directing means 150, the air-directing means of a system in accordance with the present invention can instead utilize a series of blowers (e.g. centrifugal blowers) for directing fresh air through the passageway 32 and onto the cart surfaces. In such an embodiment, the manifold employed by the liquid medium-directing means would be free to direct (or spray) a liquid medium onto the carts 23 as the manifold is returned along the passageway from the exit 42 toward the entrance 40 thereof. Accordingly, the aforedescribed embodiments are intended for the purpose of illustration and not as limitation.

1. A system for sanitizing an item, such as a grocery store shopping cart or a stroller, for re-use, the system comprising:
means defining a passageway having an entrance through which an item to be sanitized enters the passagway-defining means, an exit through which the item exits the passageway-defining means, and a passageway station disposed between the entrance and the exit of the passageway;
means for conveying the item between the entrance and the exit of the passageway so that the item is conveyed through the passageway station; and
means associated with the passageway station for directing beams of light toward the item as the item is positioned within the passageway station so that the surfaces which are struck by the beams of light are sanitized thereby.

2. The system as defined in claim 1 wherein the beams of light directed by the light beam-directing means include wavelengths of light which are in the ultraviolet (UV) range.

3. The system as defined in claim 2 wherein the system further includes means associated with the light beam-directing means for emitting beams of light in a pulsating manner.

4. The system as defined in claim 1 wherein the means for conveying is adapted to halt the advance of the item through the passageway of the passageway-defining means when the item is positioned within the passageway station so that the light beams directed from the light beam-directing means act upon the item while it is disposed in a stationary condition within the passageway station.

5. The system as defined in claim 1 wherein the passageway of the passageway-defining means includes interior surfaces which are highly reflective to the directed light beams so that the capacity of the directed light beams to sanitize the surfaces of an item positioned within the passageway station is enhanced.

6. The system as defined in claim 1 wherein the passageway of the passageway-defining means includes stationary sidewalls which are disposed on opposite sides of the passageway, and the passageway-defining means further includes:
an entrance door mounted adjacent the entrance of the passageway for movement relative to the sidewalls of the passageway between an opened condition at which an item can be conveyed through the passageway entrance and a closed condition; and
an exit door mounted adjacent the exit of the passageway for movement relative to the sidewalls of the passageway between an opened condition at which an item can be conveyed through the passageway exit and a closed condition.

7. The system as defined in claim 6 wherein the passageway includes a top which extends between the passageway sidewalls and each of the entrance and exit doors are in the form of roll-up doors which are mounted upon the passageway-defining means adjacent the top thereof for movement between the opened and closed condition.

8. The system as defined in claim 7 wherein the operation of the light beam-directing means is coordinated with the condition of the entrance and exit doors so that the light beam-directing means are operable when the entrance and exit doors are in the closed condition.

9. The system as defined in claim 1 wherein the passageway station possesses such a length as measured between the entrance and exit of the passageway so that a plurality of items to be sanitized can be positioned end-to-end within the passageway station so that a plurality of items can be sanitized simultaneously within the passageway station.

10. The system as defined in claim 1 further including means associated with the passageway station for directing a liquid medium toward the item.

11. The system as defined in claim 10 further including means for re-circulating the liquid medium through the system so that after the liquid medium has been directed toward the item, the liquid medium is collected for purposes of being subsequently directed toward an item positioned within the passageway station.

12. The system as defined in claim 11 further including means associated with the re-circulating means for sanitizing the liquid medium which is collected for purposes of being subsequently directed toward an item positioned within the passageway station.

13. The system as defined in claim 12 wherein the associated means for sanitizing the liquid medium includes means for directing beams of ultraviolet (UV) light in a pulsating manner toward the collected liquid medium so that the liquid medium is sanitized by the pulsating beams of UV light.

14. The system as defined in claim 10 wherein the means associated with the passageway station for directing a liquid medium toward the item includes a plurality of spray nozzles through which the liquid medium is directed, and the plurality of spray nozzles are mounted for movement along the length of the passageway station, and the system further includes means for moving the plurality of spray nozzles along the length of the passageway while the liquid medium is being directed toward the item.

15. The system as defined in claim 14 further including means associated with the passageway station for directing air toward the item after the item has been exposed to the liquid medium directed thereat.

16. A method for sanitizing an item, such as a grocery store shopping cart or child stroller, for re-use, the method comprising the steps of:
providing a passageway having an entrance through which an item to be sanitized enters the passageway-defining means, an exit through which the item exits the passageway-defining means, and a passageway station disposed between the entrance and the exit of the passageway;
conveying the item between the entrance and the exit of the passageway so that the item is conveyed through the passageway station; and
directing beams of ultraviolet (UV) light toward the item as the item is positioned within the passageway station so that the surfaces which are struck by the beams of UV light are sanitized thereby.

17. The method as defined in claim 16 wherein the beams of UV light directed toward the item during the directing step are directed thereat in a pulsating manner.

18. The method as defined in claim 17 wherein the step of conveying is halted when the item is positioned within the passageway station so that the surfaces of the item which are sanitized by the light beams directed from the light beam-directing means are acted thereon while the item is disposed in a stationary condition within the passageway station.

19. The method as defined in claim 18 further including a step of directing a liquid medium toward the item while the item is positioned within the passageway station.

20. The method as defined in claim 19 wherein the step of directing a liquid medium is followed by a step of collecting the liquid medium which has been directed toward the item so that the liquid medium can subsequently be directed toward an item positioned within the passageway station.

21. The method as defined in claim 20 wherein the step of collecting is followed by a step of directing beams of UV light in a pulsating manner toward the collected liquid medium so that the liquid medium is sanitized by the pulsating beams of UV light.

22. The method as defined in claim 19 wherein the step of directing a liquid medium is followed by a step of directing air toward the item for drying purposes.

23. A system for cleaning an item, such as a grocery store shopping cart or child stroller, for re-use, the system comprising:
means defining a passageway having an entrance through which an item to be cleaned enters the passageway-defining means, an exit through which the item exits the passageway-defining means, and a passageway station disposed between the entrance and the exit of the passageway;
means for conveying the item between the entrance and the exit of the passageway so that the item is conveyed through the passageway station;
means associated with the passageway station for directing a liquid medium toward the item as the item is positioned within the passageway station so that the surfaces which are struck by the liquid medium are cleaned thereby; and
means connected to the passageway-defining means for re-circulating the liquid medium through the passageway station so that after the liquid medium has been directed toward the item, the liquid medium is collected for purposes of being subsequently directed toward an item positioned within the passageway station.

24. The system as defined in claim 23 further including means associated with the re-circulating means for directing beams of ultraviolet (UV) light in a pulsating manner toward the collected liquid medium so that the liquid medium is sanitized by the pulsating beams of UV light.

25. The system as defined in claim 19 wherein the means for directing a liquid medium toward the item includes a plurality of spray nozzles through which the liquid medium is directed, and the plurality of spray nozzles are mounted for movement along the length of the passageway station, and the system further includes means for moving the plurality of spray nozzles along the length of the passageway section while the liquid medium is being directed toward the item.
